(19) United States
(12) Patent Application Publication
(10) Pub. No.: US 2002/0192266 A1
(43) Pub. Date: Dec. 19, 2002

(54) Therapeutic and nutritive dietary composition and method of use

(76) Inventor: Douglas C. Miles, Salt Lake City, UT (US)

Correspondence Address: Bruce E. Harang, PO Box 19161, Asheville, NC, 28815-1161, US

(21) Appl. No.: 10/147,365

(22) Filed: May 16, 2002

Related U.S. Application Data
(60) Provisional application No. 60/292,686, filed on May 21, 2001.

(51) Int. Cl. A61K 47/00

(52) U.S. Cl. 424/439

(57) ABSTRACT

The present invention realizes therapeutic benefits, for developing and maintaining a healthful body and mind, for improving memory, and for creating additional muscle without exercise, by utilizing natural whole raw seeds taken orally.

This application claims the benefit of the filing date of co-pending provisional patent application serial No. 60/292,686 filed on May 21, 2001 under 35 U.S.C. 119(e).

### FIELD OF THE INVENTION

The invention relates generally to a dietary food composition and method of use that provides an all-natural improvement in general health. More specifically, the invention relates to a food composition and method of use that provides for the decrease in body fat, increase in muscle tone, muscle development, and improved skin condition.

### BACKGROUND OF THE INVENTION

Numerous attempts have been made to improve the general health and well being of humans. These attempts have been directed at a person's general well being as well as preventing, treating, and correcting disorders due to aging and/or dietary deficiencies. Disorders due to aging may result if the body produces too much or too little of certain enzymes or hormones, thereby affecting the body's metabolism. A number of references teach that some such disorders are treated or corrected by supplementing missing natural elements, which are ordinarily not found in the average diet. Through the use of a daily supplement, supplying these missing vitamins and natural elements, the symptoms of various disorders may improve or disappear entirely. This piecemeal approach however creates the need for numerous supplements and even then does not provide for improving the overall general well-being or correcting the broad spectrum of disorders brought on by aging. In U.S. Pat. No. 4,548,831 issued to Iwamura on Oct. 22, 1985, and U.S. Pat. No. 6,004,558 issued to Thurn et al. on Dec. 21, 1999, seeds have been used as a source of missing natural elements by extracting portions of the seed components but not through the use of whole raw seeds. Instead, the use of extracts and insoluble portions of seeds have been utilized as a part of compositions containing additional chemicals. The use of seeds or seed extracts in combination with other ingredients or procedures to correct specific disorders has also been advocated. For example, U.S. Pat. No. 5,069,903 issued to Stitt on Dec. 3, 1991 discloses the use of raw flax seed which is ground to specific size and dried as a food supplement to provide the Omeg-3 complex. U.S. Pat. No. 5,112,814 issued to Sabin on May 12, 1992 discloses the use of phytic acid in the treatment of Parkinson's Disease and that phytic acid can be found in plant seeds, though the teaching is primarily directed to synthetic forms of phytic acid. U.S. Pat. No. 5,543,146 issued to Perez on Aug. 6, 1996 discloses the use of pumpkin seeds as a part of a multipart composition for alleviating the symptoms associated with enlargement of the prostate gland. U.S. Pat. No. 6,197,309 B1 issued to Wheeler on Mar. 6, 2001 discloses the use of a composition containing plant parts, plant extracts, and synthetic chemicals for the non-surgical treatment of prostate problems. U.S. Pat. No. 5,691,324 issued to Sandyk on Nov. 25, 1997 discloses the use of cooked plant or animal based foods containing the amino-acid tryptophan, in association with exposure to a pulsed magnetic field.

There still remains the need for a composition and method of use that provides for the general well being and improvement of general health in human beings.

### SUMMARY OF THE INVENTION

The inventor of the present invention has discovered that DNA, as it occurs in nature in the form of whole raw seeds, can be used as a food with the beneficial effect of improving the overall general well-being and health. This can be accomplished by eating whole raw seeds as a regular diet which, if desired, can be supplemented with other foods.

The present invention is directed to a therapeutic and nutritive dietary composition comprising whole raw seeds, and a method of using same, for alleviating the symptoms associated with aging and loss of general overall health.

More particularly, the invention resides a therapeutic and nutritive dietary composition comprising raw seeds and spring wheat bran masticated and soaked with digestive juices in the mouth. The composition is taken initially twice a day as the sole food intake, and thereafter taken once a day in conjunction with portions of vegetables, pastas, breads, and the like. Preferably no meat or other animal protein of any kind is taken with the present invention.

One object of the invention is to provide a therapeutic and nutritive dietary composition that provides for the correction of health conditions attributed to aging.

Another object of the invention is to provide improved health and general well-being without any adverse side effects, and that has no interactions with any medications.

A further object of the invention is to provide for weight loss without creating a constant hunger sensation.

Yet another object of the invention is to provide for improving skin condition, and provide a youthful and healthful appearance.

Still another object of the invention is to provide for improved metal faculties, and increase physical and mental stamina.

Yet still another object of the invention is to provide for increase of muscle mass without need for exercise.

Beneficial effects of the present invention include a sense of appetite satisfaction, ability to eat and enjoy sugar, lower the amount of body fat, improve muscle tone and development, and improve the overall appearance of the skin, as well as improvement of both mental and physical stamina and endurance.

A complete understanding of the invention may be obtained from the detailed description that follows.

### BEST MODE FOR CARRYING OUT THE INVENTION

The composition of the present invention comprises a therapeutically effective amount of editable raw seeds, preferably from about four to six tablespoons of editable raw seeds, and most preferably in combination with a therapeutically effective amount of uncooked spring wheat bran, most preferably about four tablespoons of uncooked spring wheat bran. The composition is administered initially twice a day orally and masticated and soaked with digestive juices in the mouth. The composition is taken daily until the desired therapeutic results are realized. It may be taken once a day indefinitely to maintain the desired therapeutic results. Preferably, no meat or animal protein of any kind is eaten during the use of the composition of the present invention.

One preferred embodiment of the present invention comprises a therapeutically effective amount of edible raw seeds administered orally and masticated and soaked with digestive juices in the mouth.

Another preferred embodiment of the present invention comprises, a therapeutically effective amount of edible raw seeds, in combination with a therapeutically effective amount of uncooked spring wheat bran, administered orally and masticated and soaked with digestive juices in the mouth. The composition is taken daily until the desired therapeutic results are realized. It may be taken indefinitely to maintain the desired therapeutic results. Preferably, no meat or animal protein of any kind is eaten during the use of the composition of the present invention.

An alternate composition of the present invention comprises a therapeutically effective amount of edible raw seeds as a source of DNA and may be the only food intake. The amount of seeds eaten on a daily basis may be adjusted to meet the needs of the individual person. The composition is administered orally and masticated and soaked with digestive juices in the mouth. The composition is taken daily until the desired therapeutic results are realized. It may be taken indefinitely to maintain the desired therapeutic results. Preferably, no meat or animal protein of any kind is eaten during the use of the composition of the present invention.

The phrase “a therapeutically effective amount” means the amount of the composition that provides a therapeutic benefit in the improvement of general health, including the creation of additional muscle and improvement of skin condition, and the reduction of stored body fat.

The editable raw seeds suitable for use in the present invention include any plant seed that is editable by humans in its raw form. Suitable editable raw seeds include, for example, sesame, flax, sunflower, caraway, filberts, pine nuts, pumpkin, fennel, brown rice, figs, cunnin, and mustard.

The method of use of the present invention preferably includes abstaining from ingesting any meat or animal protein of any kind during the period the composition of the present invention is being utilized. Additionally, during the about the first eight to twelve weeks of using the composition of the present invention no other food is ingested. After this initial period of use daily portions of vegetables, pastas, breads, and other non-meat containing foods may be eaten in addition to the composition of the present invention.

Every complete seed contains a complete set of DNA and enough nourishment to produce, upon germination, a root, a staff or stem, and a leaf exactly the same as the parent plant. The DNA provides the necessary information to reproduce new cells exactly like the cells of the seed's parent.

While not ascribing to any one particular theory, it is believed that the ability of plant DNA to reproduce new living cells exactly like the parent cells can be utilized by the human body through ingestion of the raw editable seed DNA. It has been theorized that the ingested raw editable seed DNA nucleic acid changes sugar in the body to nucleic protein and this nucleic protein produces new body cells. The use of sugar by the nucleic acid draws sugar and fat stored in the body to produce additional nucleic protein. This process reduces the amount of stored fat in the body. The production of additional nucleic protein causes the production of additional new cells resulting in improved condition of the skin, muscles, and organs. The production of new cells can include muscle development and an increase in muscle bulk without exercise.

The benefits of the composition of the present invention include appetite satisfaction thus precluding the sensation of hunger associated with general dieting for body fat lose, ability to eat high sugar containing foods with no adverse effects to body weight, loss of excess body fat, increased muscle tone and muscular development, and improved skin tone and appearance. Additionally, improved metal alertness, improved memory, and increased ability for physical exertion were observed.

### PATIENT EXAMPLES

### Example 1

A typical patient's experience involves an 81 year old man who noted lose of muscle bulk, aged skin, excess body fat, and lose of mental alertness. He started taking the nutritive composition of the present invention in October, 1998. He followed the method and composition of the present invention as his sole food intake for the first 6 weeks. Thereafter he supplemented the composition of the present invention with daily portions of vegetables, pasta, and bread, while totally abstaining from all animal protein. The patient experienced completely satisfied appetite and no feeling of hunger. In the first several months the patient's excess body fat disappeared with a weight loss of approximately ten pounds. After about four months of following the present invention, the patient started regaining lost muscle tissue and now after three and a half years of following the present invention the patient's muscle volume has returned to levels associated with men much younger. During this period no exercise or drugs were used to increase muscle bulk or tone. The patient's skin has lost all aging blemishes including one small cancer type blemish on his right forehead. Before starting to follow the present invention the patient had experienced reduced mental alertness and dozing during normal daily activities. Currently, these symptoms have substantially disappeared. Before following the present invention the patient had had to stop shoveling snow due to stress and fatigue. After approximately two years of following the present invention the patient is again shoveling snow without experiencing either chronic fatigue or stress. This patient continues to practice the invention after three and a half years.

### Example 2

A 36 year old male was started practicing the present invention. He was diagnosed with high blood pressure, diabetes, liver dysfunction, and thyroid dysfunction. He had been on medication under a Doctor's care for many months. Traditional medical care including drugs had not resulted in improvement of his ailments. After five months of following the present invention his Doctor reported to him that all of his medical test results were now back to normal.

Although this invention has been described with reference to a particular embodiment, it is not to be construed in a limiting sense. Many variations and modifications will now occur to those skilled in the art.

What is claimed is:

1. A therapeutic and nutritive dietary composition for the improvement or alleviating of the symptoms associated with aging and loss of general overall health comprising:
a therapeutically effective amount of edible raw seeds, and
an amount of digestive juices effective in soaking said therapeutically effective amount of edible raw seeds.

2. The therapeutic and nutritive dietary composition as claimed in claim 1 wherein, said therapeutically effective amount of edible raw seeds comprises from about four to about six tablespoons.

3. The therapeutic and nutritive dietary composition as claimed in claim 1 wherein, said edible raw seeds are selected from the group comprising sesame, flax, sunflower, caraway, filberts, pine nuts, pumpkin, fennel, brown rice, figs, cunnin, and mustard.

4. The therapeutic and nutritive dietary composition as claimed in claim 1 wherein, said composition is taken twice a day with no other food intake for an initial period of six to eight weeks, and then taken once a day with the addition of other food.

5. The therapeutic and nutritive dietary composition as claimed in claim 1 wherein, said composition additionally comprises a therapeutically effective amount of uncooked spring wheat bran.

6. The therapeutic and nutritive dietary composition as claimed in claim 5 wherein, said therapeutically effective amount of uncooked spring wheat bran comprises about four tablespoons.

7. The therapeutic and nutritive dietary composition as claimed in claim 5 wherein, said composition is taken twice a day with no other food intake for an initial period of six to eight weeks, and then taken once a day with the addition of other food.

6. A method for the improvement or alleviating of the symptoms associated with aging and loss of general overall health comprising:
orally administering a therapeutically effective amount of the composition of claim 1, and
masticating said composition for sufficient time to completely soak said composition with digestive juices.

7. The method as claimed in claim 6 wherein, no meat or animal protein is ingested during the course of using said method.

8. The method as claimed in claim 6 wherein, said composition is taken twice a day with no other food for an initial period of six to eight weeks and then taken once a day with the addition of other food.

9. A method for the improvement or alleviating of the symptoms associated with aging and loss of general overall health comprising:
orally administering a therapeutically effective amount of the composition of claim 5, and
masticating said composition for sufficient time to completely soak said composition with digestive juices.

10. The method as claimed in claim 9 wherein, no meat or animal protein is ingested during the course of using said method.

11. The method as claimed in claim 9 wherein, said composition is taken twice a day with no other food for an initial period of six to eight weeks and then taken once a day with the addition of other food.
